(19) United States
(12) Patent Application Publication
(10) Pub. No.: US 2021/0196760 A1
(43) Pub. Date: Jul. 1, 2021

(54) PHARMACEUTICAL COMPOSITION FOR TREATING CARTILAGE DAMAGE, COMPRISING NASAL SEPTUM CHONDROCYTES

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Jung Ho CHUN, Seoul (KR); Sun hwa PARK, Seoul (KR); Jung Yeon LIM, Seoul (KR); Mi Hyun LIM, Gyeonggi-do (KR)

(21) Appl. No.: 17/268,635

(22) PCT Filed: Aug. 16, 2019
(86) PCT No.: PCT/KR2019/010438

§ 371 Date: Feb. 16, 2021

(30) Foreign Application Priority Data
Aug. 16, 2018 (KR) 10-2018-0095658
Aug. 14, 2019 (KR) 10-2019-0099786

(51) Int. Cl. A61K 35/32; A61K 38/39; A61P 19/04; A61K 38/17

(52) U.S. Cl. CPC A61K 35/32; A61K 38/1709; A61P 19/04; A61K 38/39

(57) ABSTRACT

The present invention relates to a pharmaceutical composition for treating cartilage damage, the composition comprising nasal septum chondrocytes (NSCs) as an active ingredient, and a method for producing the NSCs into a spheroidal shape. The NSCs enable the expression of type II collagen which is a constituent component of cartilage, and SOX9 which is involved in chondrogenic differentiation, and an excellent cartilage treatment effect was shown as a result of administrating spheroidal NSCs to an animal model of cartilage damage, and thus the pharmaceutical composition and the method for producing the NSCs, according to the present invention, may be useful employed in the field of autologous chondrocyte implantation.

### TECHNICAL FIELD

The present invention relates to a pharmaceutical composition for treating cartilage damage, which includes nasal septum chondrocytes (NSCs) as an active ingredient, and a method of producing the NSCs in a spheroidal shape.

### BACKGROUND ART

Articular cartilage consists of dense and elastic connective tissue, and is located at several connection sites in the skeleton. Cartilage is connected to a bone, and its surface is in contact with another cartilage as a connecting part. Cartilage is tissue that does not contain blood vessels (avascular tissue) and nerves, generally consists of single cell-type basic chondrocytes and is synthesized as an extracellular matrix (ECM).

Articular cartilage treatment still remains an unsolved problem in modern medicine, and even though there are treatment methods, many problems remain. One of the biggest problems is that articular cartilage has a very low self-repair capacity. To solve this problem, regenerative medicine has begun to be developed, and cartilage tissue engineering repairs tissue through biological replacement.

An autologous chondrocyte implantation (ACI) technique is used when a large surface area of the articular cartilage is damaged, and autologous chondrocytes to be implanted are obtained by biopsy of a small portion of healthy articular cartilage and isolation with an enzyme. However, the commonly known naturally occurring autologous chondrocytes have limitations.

With in vitro expansion culture, there is a limit in chondrocyte redifferentiation, and chondrogenic potential decreases with the age of a donor, and cell proliferation is important due to the characteristic of ACI using a large quantity of cells, and articular chondrocytes are known to have very low proliferation power. Human NSCs can compensate for such problems of human articular chondrocytes. Human NSCs have a higher proliferation rate than human articular chondrocytes, also have high in vitro and in vivo chondrogenic capacity, and do not decrease depending on the age of a donor.

However, whereas the development of a therapeutic agent for treating articular damage using NSCs having such excellent effects is insufficient, the clinical demand for complete regeneration of damaged cartilage is rapidly increasing, and this demand has a tendency to further increase in this time of an increasingly aging society.

### DISCLOSURE

### Technical Problem

The inventors confirmed that nasal septum chondrocytes (NSCs) express collagen type 2 and SOX9, and as a result of administering NSCs cultured in a spheroidal shape into a cartilage damaged animal model, confirmed that NSCs have superior cartilage treatment capacity to articular chondrocytes, and thus the present invention was completed.

Therefore, the present invention is directed to providing a pharmaceutical composition for treating cartilage damage, which includes NSCs as an active ingredient.

The present invention is also directed to providing a method of producing NSCs for treating cartilage damage, which includes:

a) isolating nasal septum chondrocytes (NSCs) from nasal septum tissue;

b) culturing the NSCs isolated in Step a) in a cell culture container that can culture cells in a spheroidal shape using a medium for cell culture containing bovine serum albumin (BSA); and

c) recovering spheroid-shaped NSCs cultured in the cell culture container.

The present invention is also directed to providing a method of treating cartilage damage, which includes administering a pharmaceutical composition including NSCs as an active ingredient into a subject.

The present invention is also directed to providing a use of a pharmaceutical composition including NSCs as an active ingredient for treating cartilage damage.

The present invention is also directed to providing a use of NSCs for producing a drug used to treat cartilage damage.

However, technical problems to be solved in the present invention are not limited to the above-described problems, and other problems which are not described herein will be fully understood by those of ordinary skill in the art from the following descriptions.

### Technical Solution

To attain the purpose of the present invention, the present invention provides a pharmaceutical composition for treating cartilage damage, which includes NSCs as an active ingredient.

In one embodiment of the present invention, the NSCs may have a spheroidal shape.

In another embodiment of the present invention, the NSCs may express collagen type 2 or SOX9.

The present invention also provides a method of producing NSCs for treating cartilage damage, which includes:

a) isolating NSCs from nasal septum tissue;

b) culturing the NSCs isolated in Step a) in a cell culture container that can culture cells in a spheroidal shape using a medium for cell culture containing BSA; and

c) recovering spheroid-shaped NSCs cultured in the cell culture container.

In one embodiment of the present invention, the NSCs in Step a) may be harvested after filtration with a 40 to 50-nm filter.

In another embodiment of the present invention, the method may further include mixing the spheroidal NSCs recovered in Step c) with a support.

In addition, the present invention provides a method of treating cartilage damage, which includes administering a pharmaceutical composition including NSCs as an active ingredient into a subject.

In addition, the present invention provides a use of a pharmaceutical composition including NSCs as an active ingredient for treating cartilage damage.

In addition, the present invention provides a use of NSCs for producing a drug used to treat cartilage damage.

### Advantageous Effects

The present invention relates to a pharmaceutical composition for treating cartilage damage, which includes nasal septum chondrocytes (NSCs) as an active ingredient, and a method of producing the NSCs in a spheroidal shape, in which the NSCs express collagen type 2, which is a constituent of cartilage, and SOX9 involved in chondrogenic differentiation, and as a result of administering spheroidal NSCs into a cartilage-damaged animal model, the NSCs were engrafted to a damaged site, and thus exhibited a superior cartilage treatment effect to the result of the administration of spheroidal articular chondrocytes. The pharmaceutical composition of the present invention and the method of producing spheroidal NSCs can be effectively used in an autologous chondrocyte implantation (ACI) field.

### DESCRIPTION OF DRAWINGS

FIG. 1 is a result of comparing collagen type 2 and SOX9 expression by comparing 8 types of human nasal septum chondrocytes (hNSCs) and human inferior turbinate-derived mesenchymal stem cells (hTMSCs) using western blotting.

FIG. 2 show the result of staining cultured cells using hematoxylin & eosin (H&E), Alcian blue and Masson's Trichrome to compare morphological differences between 2D cell culture and 3D cell culture.

FIG. 3 shows the result of confirming a survival rate through Live & Dead staining after culture of human NSCs (hNSCs) and articular chondrocytes (ACs) by spheroidal culture.

FIG. 4 shows a schematic experimental process for confirming a cartilage regeneration capacity in a cartilage-damaged animal model.

FIG. 5 shows a method of preparing a cartilage-damaged animal model and a method of implanting cartilage.

FIG. 6 shows the result of staining normal cartilage and cartilage at a defect site of a cartilage-damaged animal model with H&E, Alcian blue, Safranin O and Trichrome.

FIG. 7 shows the result of showing the difference in cartilage regeneration capacity when hNSCs and ACs in a spheroidal pellet- or non-pellet-type cell state are implanted into cartilage-damaged animal models.

FIG. 8 shows the result of confirming that cartilage is engrafted by culturing human NSCs in a spheroidal pellet and implanting it into a cartilage-damaged animal model.

FIG. 9 shows the result of verifying cell engraftment after an injectable NSC therapeutic agent is administered into a cartilage-damaged animal model.

FIG. 10 shows the result of histological analysis of knee cartilage after human NSC spheroids or human articular chondrocyte spheroids are mixed with collagen and then the mixture is implanted into a cartilage-damaged animal model.

FIG. 11 shows the result of confirming the morphological difference of knee cartilage after human NSCs and human NSC spheroids are implanted into cartilage-damaged animal models, respectively.

### MODES OF THE INVENTION

The inventors confirmed that nasal septum chondrocytes (NSCs) express collagen type 2 and SOX9, and as a result of administering NSCs cultured in a spheroidal shape into a cartilage damaged animal model, confirmed that NSCs have superior cartilage treatment capacity to articular chondrocytes, and thus the present invention was completed.

In one embodiment of the present invention, a method of isolating human NSCs from nasal septum tissue and a culturing method thereof were confirmed (see Example 1).

In another embodiment of the present invention, western blotting was performed to confirm that collagen type 2, which is a constituent of cartilage, and SOX9 involved in chondrogenic differentiation are expressed in human NSCs (see Example 2).

In still another embodiment of the present invention, a method of producing human NSCs in a spheroidal shape was confirmed (see Example 3).

In yet another embodiment of the present invention, in 3D culture, such as spheroidal culture, it was confirmed that cells are formed in a spherical shape (see Example 4).

In yet another embodiment of the present invention, it was confirmed that the viability of spheroid-shaped NSCs is superior to that of spheroid-shaped articular chondrocytes (see Example 5).

In yet another embodiment of the present invention, a cartilage-damaged animal model was established, and a spheroid pellet made using NSCs was administered into a damaged joint site of the cartilage-damaged animal model, thereby confirming pellet engraftment (see Examples 6 and 7).

In yet another embodiment of the present invention, as a result of administering spheroid-shaped NSCs into a damaged joint site of a cartilage-damaged animal model, it was confirmed that the cells were well grafted (see Example 8).

In yet another embodiment of the present invention, when spheroid-shaped human NSCs were administered into a cartilage-damaged animal model, compared to when spheroid-shaped human articular chondrocytes were administered, it was confirmed that a smoother cartilage regenerating effect was shown (see Example 9). In yet another embodiment of the present invention, when spheroid-shaped human NSCs were administered into a cartilage-damaged animal model, compared to when human NSCs were administered, it was confirmed that a smoother cartilage regenerating effect was shown (see Example 10).

From the above-described results of the embodiments, as it was confirmed that the spheroid-shaped NSCs according to the present invention can heal cartilage damage, the spheroid-shaped NSCs can be used to treat cartilage damage.

Therefore, the present invention provides a pharmaceutical composition for treating cartilage damage, which includes NSCs as an active ingredient.

The term “treatment” used herein refers to all actions involved in alleviating or beneficially changing symptoms of cartilage damage by administration of the pharmaceutical composition according to the present invention.

The term “chondrocytes” used herein are cells that are present in the chondrin of the matrix of cartilage, synthesize and secrete the matrix of cartilage, and have a well-developed rough endoplasmic reticulum and Golgi apparatus. The appearance is consistent with the shape of the cartilage lumen, and the chondrocytes are present in a long elliptical or flat shape under the cartilage membrane and on an articular cartilage surface, and in a semicircular or polygonal shape in the deeper part. There is a complex of polysaccharides or proteins binding to the cell membrane of a chondrocyte. Since this complex sterically binds to a polysaccharide or fibers of a matrix, a chondrocyte floats in the matrix. In the present invention, the chondrocyte is a concept that also includes chondroprogenitor cells, which are cells whose differentiation direction has been determined as chondrocytes.

The term “NSC” used herein refers to a chondrocyte that forms the front part of the nasal septum, which is the partition dividing the nasal cavity into left and right sides. The NSC of the present invention may be isolated from nasal septum cartilage tissue discarded in nasal septoplasty, which is one of the most frequent surgeries or nasal septum cartilage tissue obtained by simple biopsy under local anesthesia, and as a non-weight bearing donor site, does not have complications caused by local anesthesia. In addition, while there is no report on the number of chondrocytes isolated from nasal septum tissue, compared to articular chondrocytes, a larger number of chondrocytes can be secured, and chondrocytes isolated from nasal septum cartilage, which is hyaline cartilage, have a higher cell proliferation capacity and an excellent capacity to produce a cartilage-specific extracellular matrix in in vitro culture, compared with articular chondrocytes.

The term “damage” used herein refers to any phenomenon in which the normal structure of tissue is morphologically destroyed regardless of cause.

The pharmaceutical composition according to the present invention may include NSCs as an active ingredient, and a pharmaceutically acceptable carrier. The pharmaceutically acceptable carrier is generally used in formulation, and includes saline, distilled water, Ringer's solution, buffered saline, cyclodextrin, a dextrose solution, a maltodextrin solution, glycerol, ethanol, liposomes, etc., but the present invention is not limited thereto. If needed, the pharmaceutically composition may further include other conventional additives including an antioxidant, a buffer, etc. In addition, by additionally adding a diluent, a dispersant, a surfactant, a binder or a lubricant, the pharmaceutical composition may be formulated as an injectable form such as an aqueous solution, an emulsion or a suspension, a pill, a capsule, a granule or a tablet. Suitable pharmaceutically acceptable carriers and their formulations may be formulated according to each ingredient using a method disclosed in the Remington's Pharmaceutical Science. The pharmaceutical composition of the present invention is not limited in dosage form, and thus may be formulated as an injection, an inhalant, a dermal preparation for external use, or an oral preparation.

The pharmaceutical composition of the present invention may be administered orally or parenterally (e.g., intravenously, subcutaneously, percutaneously, nasally or intratracheally) according to a desired method, and a dose of the pharmaceutical composition of the present invention may be selected according to a patient's condition and body weight, severity of a disease, a dosage form, an administration route and duration by those of ordinary skill in the art.

The composition according to the present invention is administered at a pharmaceutically effective amount. In the present invention, the “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

Specifically, the effective amount of the composition according to the present invention may be changed according to a patient's age, sex or body weight. However, the effective amount may be increased or decreased depending on the route of administration, the severity of obesity, sex, a body weight or age, and thus it does not limit the scope of the present invention in any way.

In the present invention, the NSCs may be formed in a spheroid shape.

The term “spheroid” used herein refers to a three-dimensional structure in which cells are aggregated to the extent that the cross-section can generally appear circular or elliptical, and it is apparent that this shape should be determined by considering the characteristics of cells or a cell aggregate, and does not refer to a perfect spheroidal or spherical shape.

In the present invention, the NSCs may express collagen type 2 or SOX9.

Regarding the term “collagen type 2” used herein, collagen is the fibrous protein that is mainly present in the bone and skin of an animal, and found in cartilage, organ membranes and hair, and also present as a fibrous solid. Collagen has a complicated striated structure when observed under an electron microscope, and does not dissolve in water, dilute acids or dilute alkalis, but when boiled, becomes gelatinous and dissolves. There are six types of collagens, and the collagen type 2 of the present invention is the main component of cartilage.

The term “SOX9” used herein is known to be involved in recognition of a CCTTGAG sequence and chondrogenic differentiation, along with other members of HMG-box class DNA-binding protein.

In another aspect of the present invention, the present invention provides a method of producing NSCs for treating cartilage damage, which includes: a) isolating nasal septum chondrocytes (NSCs) from nasal septum tissue;

b) culturing the NSCs isolated in Step a) in a cell culture container that can culture cells in a spheroidal shape using a medium for cell culture containing bovine serum albumin (BSA); and

c) recovering spheroid-shaped NSCs cultured in the cell culture container.

In Step b) of the present invention, the cell culture container is any container that can culture cells in a spheroidal shape without particular limitation, and in the following examples, StemFIT 3D (Microfit) was used, but the present invention is not limited thereto.

The NSCs in Step a) of the present invention may be acquired after harvested with a 30 to 50-nm filter, and preferably, a 40-nm filter, but the present invention is not limited thereto.

In Step b) of the present invention, the concentration of BSA may be, but not limited to, 1 to 5%, and preferably 3%.

In Step b) of the present invention, the cell culture medium and BSA may be mixed in a ratio of 2:0.5 to 1.5, and preferably 2:1, but the present invention is not limited thereto.

After Step c) of the present invention, a step of mixing the recovered spheroid-shaped NSCs and a support may be further included.

The term “support” used herein is an in vitro mimic with the properties of extracellular matrix (ECM). The morphology and function of bio tissue are maintained by an interaction with multiple types of cells and extracellular materials, and among the extracellular materials, particularly, the extracellular matrix having an organic polymer as a main component serves as a structural support of tissue and cell adhesion inducer. That is, cells should be adhered to the ECM so as to be fused to tissue and thus to perform basic functions and enable several biological regulations in vitro. In the present invention, the support is preferably a porous sponge, nanofiber, hydrogel, or collagen, but the present invention is not limited, and any ECM that can be applied clinically.

In still another aspect of the present invention, a method of treating cartilage damage, which includes administering a pharmaceutical composition including NSCs as an active ingredient into a subject, is provided.

In yet another aspect of the present invention, a use of a pharmaceutical composition including NSCs as an active ingredient for treating cartilage damage is provided.

In yet another aspect of the present invention, a use of nasal septum chondrocytes (NSCs) for producing a drug used in treatment of cartilage damage is provided.

Hereinafter, to help in understanding the present invention, exemplary examples will be suggested. However, the following examples are merely provided to more easily understand the present invention, and not to limit the present invention.

### Example 1. Isolation and Culture of NSCs

Nasal septum cartilage tissue used in this research was obtained during the procedure of nasal septoplasty, and used with the patient's content before surgery. Immediately after collection of the nasal septum cartilage tissue, the tissue sample was washed with physiological saline containing gentamicin 3 to 5 times to isolate chondrocytes.

The tissue obtained by biopsy during the surgery to isolate human NSCs was stored at 4□ in a refrigerator, and washed with phosphate buffered saline (PBS) twice before isolation of chondrocytes using the tissue. After washing, the nasal septum cartilage was cut into 1 mm³ pieces on a non-coating dish, and then the small pieces of tissue were treated with type 2 collagenase to allow an overnight reaction on a non-coating dish at 37□ in a 5% CO□ incubator (0.01 g of type 2 collagenase in 10 mL low glucose DMEM media, 10% FBS, 1% Antibiotic-Antimycotic). The isolated chondrocytes were harvested after filtration using a 40-nm filter.

The harvested chondrocytes were spun down to remove the media, and then washed with PBS. The chondrocytes were seeded in a culture dish, and cultured at 37□ in a 5% CO□ incubator.

### Example 2. Confirmation of Collagen Type 2 and SOX9 Expression in NSCs

To investigate collagen type 2 and SOX9 expression in NSCs, hNSCs and human inferior turbinate-derived mesenchymal stem cells (hTMSCs) were incubated and subjected to western blotting by the following method.

First, chondrocytes were harvested using RIPA buffer. The chondrocytes were reacted on ice for approximately 20 minutes, spun down into a pellet by centrifugation at 4□ for 20 minutes, and then only a supernatant was used. Proteins were quantified by a BCA quantification method, and denatured with SDS buffer at 100□ for 5 minutes. The quantified protein sample was subjected to electrophoresis at 80 V in a 6% polyacrylamide gel, and then transferred to a PVDF membrane. Afterward, the PVDF membrane was blocked using 5% skim milk, and then an antibody to be confirmed was attached, followed by detection of the antibody.

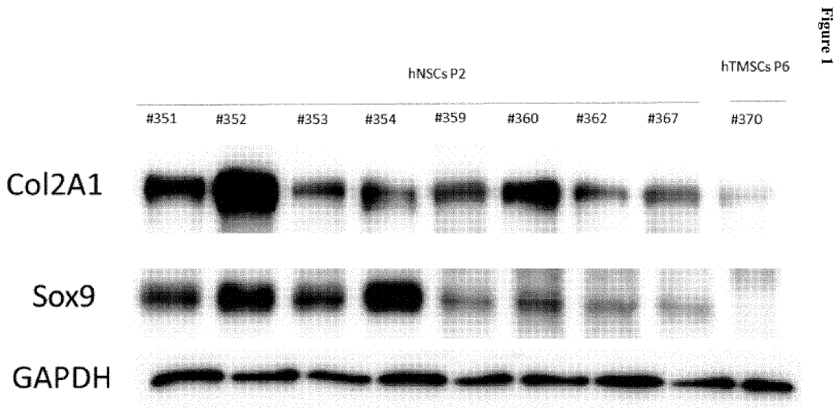

As a result, as shown in FIG. 1, it was confirmed that collagen type 2 and the SOX9 protein were not expressed in hTMSCs, but collagen type 2 and the SOX9 protein were highly expressed in hNSCs, (FIG. 1).

### Example 3. Preparation of Spheroid-Shaped NSCs

In the present invention, a cell culture container for spheroid-shaped cell culture was StemFIT 3D (Microfit), and the addition and change of all media were performed in an inner corner of the StemFIT 3D (Microfit). First, the StemFIT 3D (Microfit) was placed on a dish to be cultured, filled with 70% ethanol (EtOH), followed by pipetting to remove bubbles. After the complete removal of bubbles, 70% ethanol was suctioned from the corner of the StemFIT 3D (Microfit) using a pipette. Here, care was taken so as not to drain all of the 70% ethanol from wells so that bubbles were not generated again. A cell culture medium or 1×PBS (Welgene) was filled while each well was fully filled with 70% ethanol to prevent bubble formation and allow cell culture in the well. After observation using a microscope and suctioning, a prepared single cell was seeded in StemFIT 3D (Microfit), filtered 3% bovine serum albumin (BSA) was added to a single cell-suspended medium to have a ratio of 2:1, followed by waiting until all of the cells settled in wells of the StemFIT 3D (Microfit). Here, when the StemFIT 3D was placed on a microscope and shaken gently, the cells suspended in the medium were observed. Therefore, if washing was performed without all of the cells settled, unsettled cells were lost, and in the case of cells other than embryonic stem cells, a spheroid size could be smaller. For this reason, the inventors waited for all of the suspended cells to settle. After five minutes, if there were a large quantity of the cells between wells, the medium was gently suctioned by pipetting at the inner corner of the StemFIT 3D, and then a cell culture medium was filled such that surface tension was created inside the StemFIT 3D. Like conventional cell culture, the cells were incubated, and a first medium exchange was performed within 4 to 24 hours. When cell aggregation was observed, only a cell culture medium without BSA was added, and after 2 to 3 days, a compact spheroid was observed under a microscope. A 3D spheroid was acquired, and then mixed with a support for implantation.

### Example 4. Confirmation of Morphology of Spheroid-Shaped NSCs

To compare a morphological difference between conventional 2D cell culture and spheroid-shaped 3D cell culture, the cultured cells were stained with H&E, Alcian blue and Trichrome.

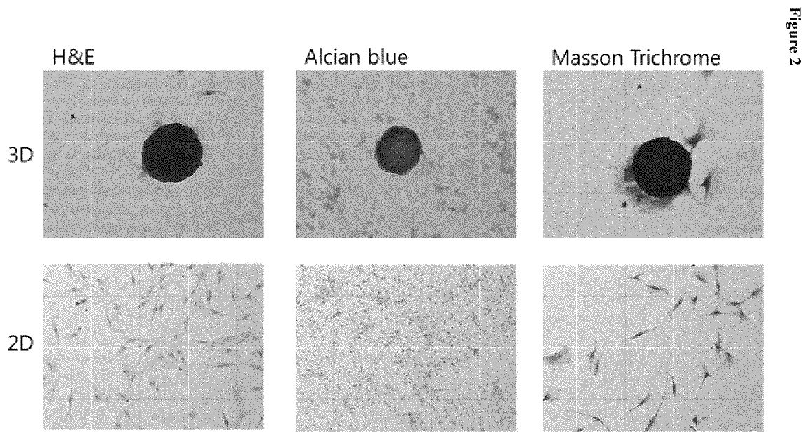

As a result, as shown in FIG. 2, it was confirmed that, in 2D culture, the cells are generally spread out and thus do not have a high density, but in 3D culture, the cells are grown in a spherical shape with a high density (FIG. 2).

### Example 5. Confirmation of Viability of Spheroid-Shaped NSCs

To analyze the viability of spheroid-shaped NSCs, spheroid culture was performed for up to 14 days, and then Live & Dead staining in which living cells show a green color and dead cells show a red color was performed.

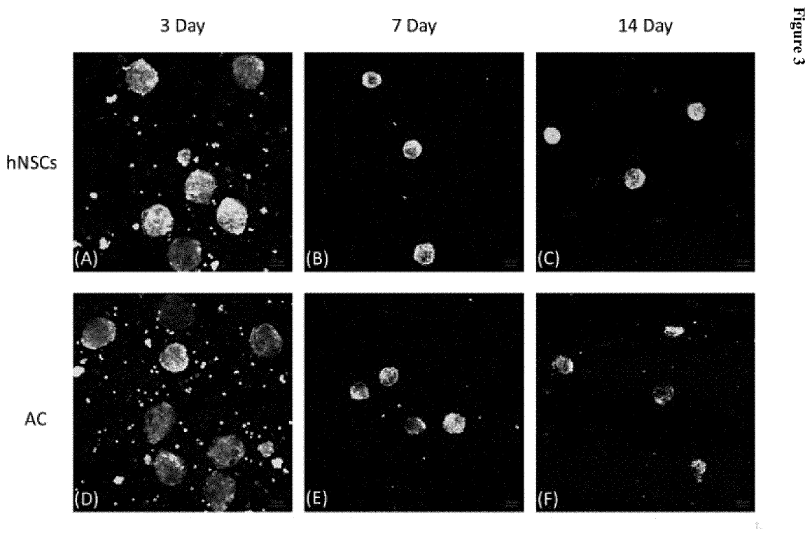

As a result, both hNSCs and human articular chondrocytes (hACs) exhibited high cell viability, and until day 14, 90% or more cell viability was shown. However, the hNSCs and the hACs showed slightly different patterns in terms of a spheroid structure. After day 7, it was confirmed that there are a very large quantity of cells in the hNSC spheroid, but in the case of the hACs, compared to hNSCs, a slightly smaller amount of cells are aggregated in the spheroid structure (FIG. 3).

### Example 6. Establishment of Cartilage-Damaged Animal Model

For experiments, SD rats (12-week-old, male) were used. According to the regulations for animal tests, 10-week-old rats were prepared and subjected to a two-week acclimation period in an animal laboratory.

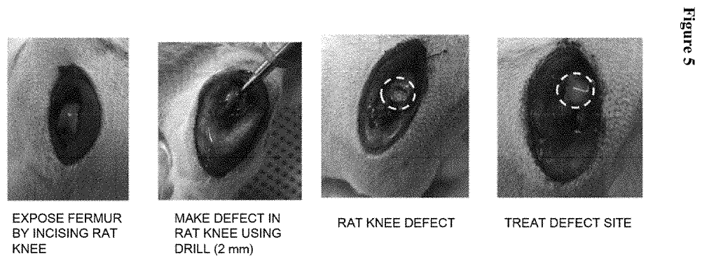

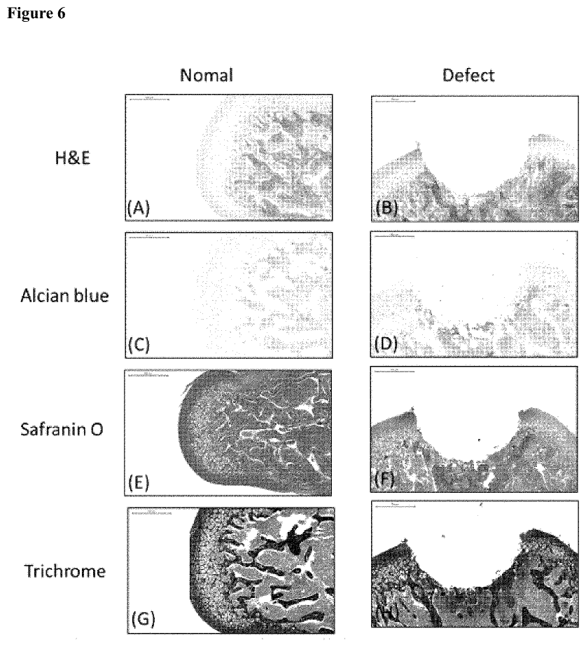

To establish a cartilage-damaged animal model, as shown in FIG. 5, a rat's knee was incised to expose a femur, and then a defect site was made in the rat's knee using a drill (2 mm). The next day, to confirm cartilage damage, as shown FIG. 6, the defect site was stained with H&E, Alcian blue, Safranin O or Trichrome.

As a result, as shown in FIGS. 6B, D, F and H, defect staining was not observed with any of these dyes, confirming that a cartilage-damaged animal model had been properly established.

### Example 7. Confirmation of Spheroid-Shaped NSC Implantation Effect

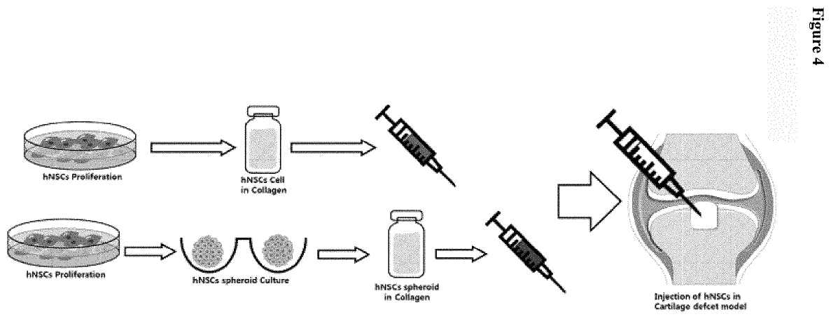

To confirm a spheroid-shaped NSC implantation effect, an experiment was conducted by the procedures shown in FIG. 4. First, spheroid pellets formed using hNSCs and hACs were implanted into cartilage-damaged rat models and harvested after 2 weeks. Cartilage sections were stained to observe a change in cartilage-damaged rat models. Glycosaminoglycan (GAG) and proteoglycan levels were determined and cartilage was detected through Alcian blue and Safranin-O staining, and collagen was detected through Trichrome staining.

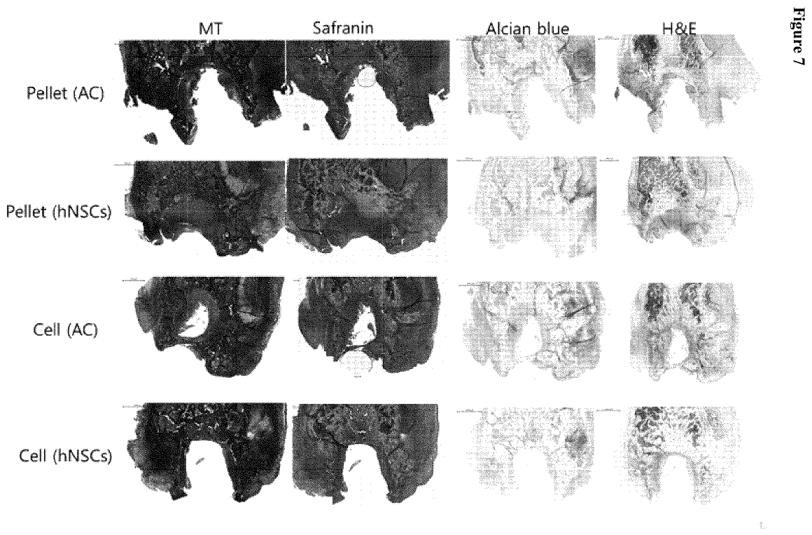

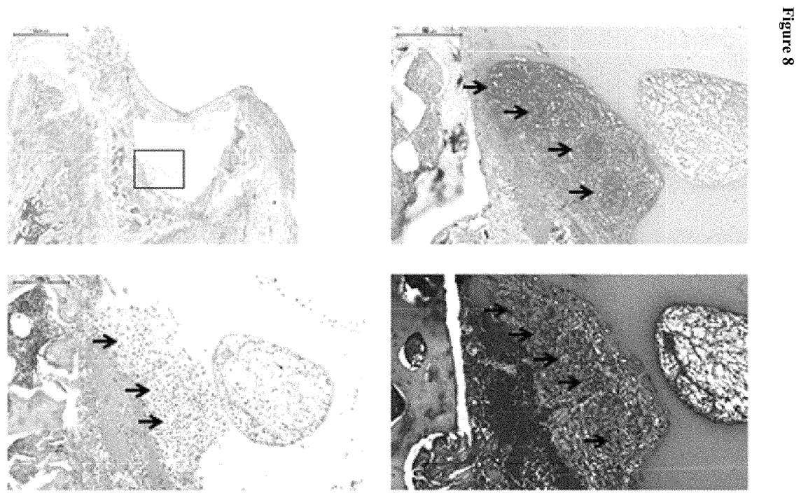

As a result, as shown in FIGS. 7 and 8, in the hNSC pellet group, pellets were engrafted, and a damaged cartilage treatment effect was confirmed through cartilage-specific staining.

### Example 8. Verification of Cell Engraftment of Injectable NSC Therapeutic Agent in Cartilage-Damaged Animal Model

As described in Example 6, a cartilage-damaged animal model was constructed, and then spheroid-shaped hNSCs and collagen were mixed and administered so that each cartilage-damaged rat model was administered with 4×10⁴ chondrocytes and 20 μl of collagen. In addition, after 4 or 8 weeks, each subject was sacrificed to obtain knee cartilage tissue, and whether the administered NSCs are well grafted was confirmed using a human nuclei antibody.

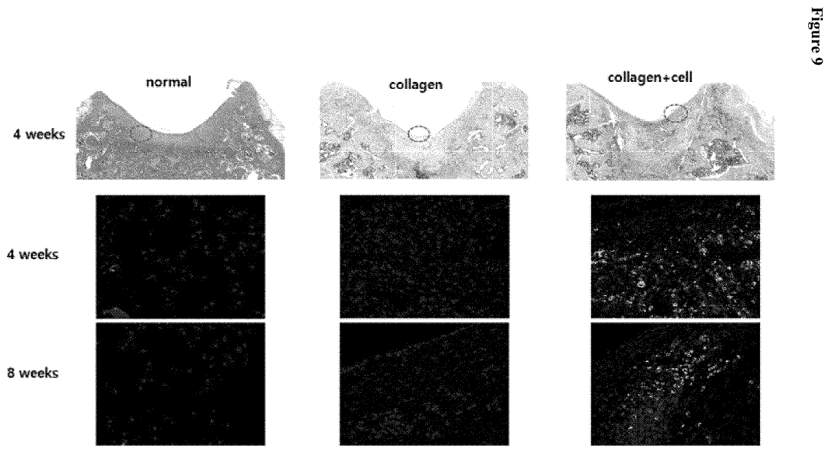

As a result, as shown in FIG. 9, cells were observed at week 4 and week 8.

### Example 9. Comparison of Cartilage Regenerating Effect Between Spheroid-Shaped hNSCs and hACs

As described in Example 6, a cartilage-damaged animal model was constructed, and then spheroid-shaped human NSCs or spheroid-shaped hACs were mixed with collagen and administered so that each cartilage-damaged rat model was administered with 4×10⁴ chondrocytes and 20 μl of collagen. For histological analysis of knee cartilage, after 4 weeks, each subject was sacrificed to obtain knee cartilage tissue, and the cells were stained with H&E, Alcian blue, Safranin O or Masson's Trichrome.

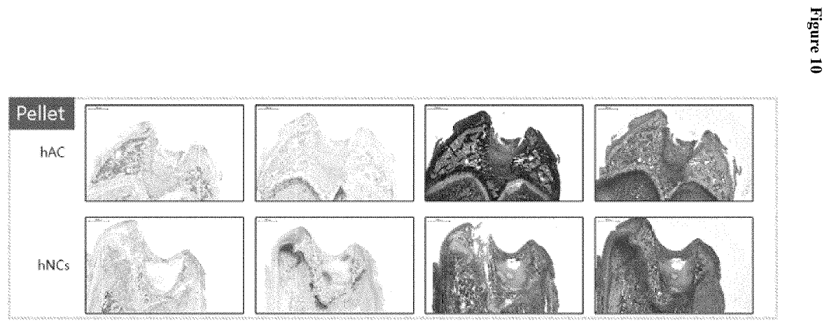

As a result, as shown in FIG. 10, it was confirmed that a smoother cartilage regenerating effect was exhibited at a damaged site of a spheroid-shaped hNSC-implanted subject, compared to a spheroid-shaped hAC-implanted damaged site.

### Example 10. Comparison of Cartilage Regenerating Effect after Implantation of hNSCs and Spheroid-Shaped hNSCs

As described in Example 6, a cartilage-damaged animal model was constructed, and then hNSCs or spheroid-shaped hNSCs were mixed with collagen and administered so that each cartilage-damaged rat model was administered with 4×10⁴ chondrocytes and 20 μl of collagen. After 8 weeks, each subject was sacrificed to obtain knee cartilage tissue, and the morphological analysis of the knee cartilage tissue was performed.

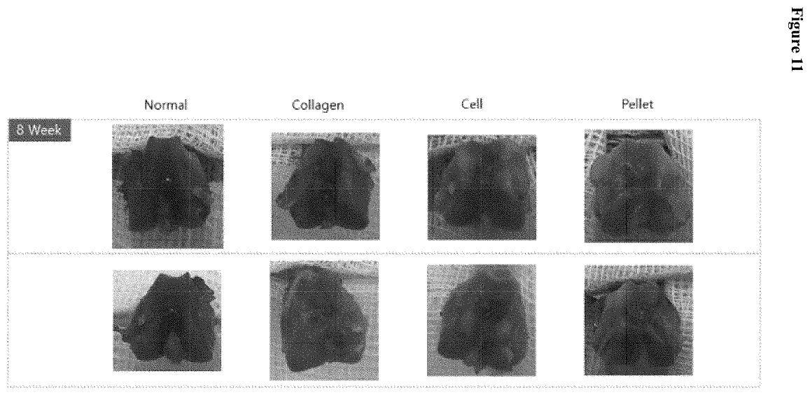

As a result, as shown in FIG. 11, it was confirmed that a smoother cartilage regenerating effect was exhibited in a spheroid-shaped hNSC-implanted subject, compared to a hNSC-implanted subject.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

According to the present invention, NSCs express collagen type 2, which is a constituent of cartilage, and SOX9 involved in chondrogenic differentiation, and as a result of administering spheroidal NSCs into a cartilage-damaged animal model, the NSCs were engrafted to a damaged site, and thus exhibited a superior cartilage treating effect to the result of the administration of spheroidal articular chondrocytes. Therefore, the pharmaceutical composition of the present invention and the method of producing spheroid-shaped NSCs are expected to be effectively used in an autologous chondrocyte implantation (ACI) field.

What is claimed is:

1. A method of treating cartilage damage, comprising administering a pharmaceutical composition comprising nasal septum chondrocytes (NSCs) as an active ingredient into a subject.

2. The method of claim 1, wherein the NSCs are formed in a spheroidal shape.

3. The method of claim 1, wherein the NSCs express collagen type 2 or SOX9.

4. A method of producing NSCs for treating cartilage damage, comprising:
a) isolating nasal septum chondrocytes (NSCs) from nasal septum tissue;
b) culturing the NSCs isolated in Step a) in a cell culture container that can culture cells in a spheroidal shape using a medium for cell culture containing bovine serum albumin (BSA); and
c) recovering spheroid-shaped NSCs cultured in the cell culture container.

5. The method of claim 4, wherein, in Step a), the NSCs are harvested after filtration with a 40 to 50-nm filter.

6. The method of claim 4, further comprising, after Step c), mixing the recovered spheroid-shaped NSCs with a support.

7. (canceled)

8. (canceled)
